(19) United States
(12) Patent Application Publication
(10) Pub. No.: US 2009/0300812 A1
(43) Pub. Date: Dec. 10, 2009

(54) Lens Assembly for a Diving Mask

(75) Inventor: Chih-Cheng Shiue, Taipei (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: QBAS CO., LTD., Taipei (TW)

(21) Appl. No.: 12/345,121

(22) Filed: Dec. 29, 2008

Related U.S. Application Data
(63) Continuation-in-part of application No. 12/136,235, filed on Jun. 10, 2008, now abandoned.

(30) Foreign Application Priority Data
Oct. 20, 2008 (TW) 97218711

(51) Int. Cl. A61F 9/00

(52) U.S. Cl. 2/15

(57) ABSTRACT

The lens assembly for a diving mask is provided. The lens assembly comprises a first lens, a second lens and an intermediate layer. The intermediate layer is made of a liquid material which is capable of being cured and bonded with the first lens and the second lens. Thereby, the lens assembly can prevent the scattering of fragments when the lens assembly is shattered. Furthermore, the second lens is preferably made of the material selected from the group consisting of cellulose acetate, cellulose propionate and the combination thereof for providing the diving mask with better anti-fog ability.

### CROSS-REFERENCES TO RELATED APPLICATIONS

This application is a continuation-in-part of application Ser. No. 12/136,235, filed on Jun. 10, 2008. This application also claims the benefits of the priority based on Taiwan Patent Application No. 097218711 filed on Oct. 20, 2008; the disclosures of which are incorporated by reference herein in their entirety.

### BACKGROUND OF THE INVENTION

1. Field of the Invention

The present invention provides a lens assembly for a diving mask.

2. Descriptions of the Related Art

Diving is a common leisure activity that allows people to enjoy a wide variety of marine creatures. Diving masks make up an important portion of the gear required to partake in diving activities, such as snorkeling and scuba diving. Diving masks protect the eyes from water by forming a barrier with the water.

Typically, the lens used in diving masks is made of glass with high transparency. Unfortunately, glass is a fragile material and can be shattered under an external impact force during diving, thus causing entry of water into the diving mask. Furthermore, there is a high potential for the fragments to injure the eyes and cause serious injury to the diver.

To avoid the lens from shattering, safety lenses have been gradually adopted for diving masks. A conventional safety lens comprises a plurality of glass layers and adhesive film layers interposed therebetween. More specifically, the glass layers and the adhesive film layers are pressed to and bonded to each other with a vacuum created therebetween. However, when the glass layers are shattered by an external force, the vacuum between the glass layers and the adhesive film layers will not exist anymore. Even if the resulting fragments are initially attached to the adhesive film layers, the fragments may still fall out from the adhesive film layers when jittered or vibrated, posing threats to users.

Furthermore, after a period of diving, fog often forms on the inner lens of the diving mask which utilizes glass as lenses and causes the view vague. The conventional method for preventing the formation of fog is smearing an additional anti-fog layer on the inner lens of the diving mask before diving. However, the effect of the anti-fog layer will gradually fade away. To maintain the anti-fog ability, smearing the anti-fog layer is constantly needed, which causes the inconvenience when using the diving mask.

Therefore, it is highly desirable in the art to provide a lens assembly that not only prevents the fragments of the lens assembly to scatter when the lens assembly is shattered, but also prevents vague view caused by the fog.

### SUMMARY OF THE INVENTION

One objective of this invention is to provide a lens assembly for a diving mask. Because of the bonding between the adhesive intermediate layer and the lens, the lens assembly is tightly secured even if broken. As a result when the lens assembly is shattered on impact, the resulting fragments will still be adhered to the intermediate layer instead of scattering.

Another objective of this invention is to provide a lens assembly for a diving mask. By using cellulose acetate or cellulose propionate as a material for the inner lens of the diving mask, the diving mask is provided with an excellent anti-fog ability.

The lens assembly disclosed in this invention comprises a first lens, a second lens, and an intermediate layer disposed between the first lens and the second lens that bonds the first lens with the second lens. The intermediate layer is made of a liquid adhesive which is capable of being cured. The material of the second lens is selected from the group consisting of polycarbonate, cellulose acetate, cellulose propionate and the combination thereof.

The detailed technology and preferred embodiments implemented for the subject invention are described in the following paragraphs accompanying the appended drawings for the people skilled in this field to well appreciate the features of the claimed invention.

### BRIEF DESCRIPTION OF THE DRAWINGS

FIG. 1 is a schematic view of a lens assembly of this invention when applied to the diving mask;

FIG. 2 is a schematic exploded view of the lens assembly of this invention; and

FIG. 3 is a schematic cross-sectional view of the lens assembly of this invention.

### DESCRIPTION OF THE PREFERRED EMBODIMENT

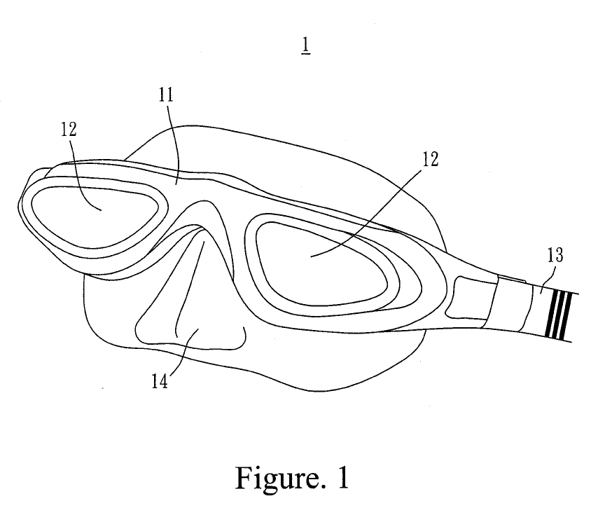

As shown in FIG. 1, the lens assembly 12 of this invention is used in a diving mask 1. The diving mask 1 further comprises a lens frame 11 disposed on a mask 14 in which at least one lens assembly 12 is installed. Herein, the diving mask 1 comprises two lens assemblies 12 both installed in the lens frame 11. The tightening belt 13 is adapted to secure the diving mask 1 on to the head of the diver, and the mask 14 is adapted to prevent any entry of liquid into the diving mask 1. The diving mask 1 of this invention may also be implemented in other similar structures that incorporate lenses. The other associated components described above are only for the purpose of illustration, rather than to limit the scope of this invention.

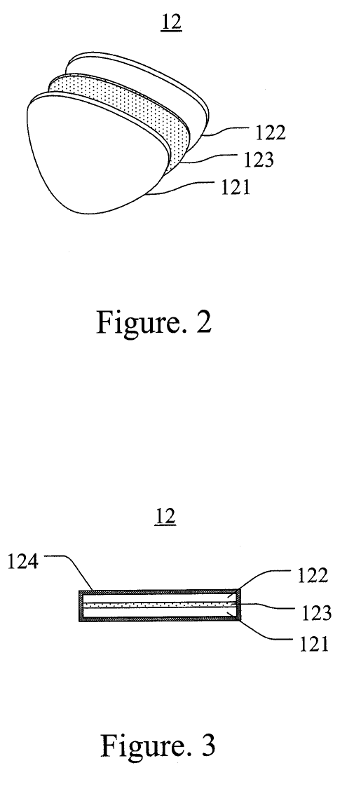

FIG. 2 illustrates the multi-layer structure of the lens assembly 12 of this invention. The lens assembly 12 comprises a first lens 121, a second lens 122 and a transparent intermediate layer 123 disposed between the first lens 121 and the second lens 122.

One of the characteristic of this invention is that the intermediate layer 123 is an adhesive layer. For example, the intermediate layer 123 may be made of a liquid adhesive which is capable of being cured. During the manufacturing process, the liquid adhesive is coated between the first lens 121 and the second lens 122 first, and then cured after the first lens 121 has been bonded with the second lens 122, thus creating an adhesive intermediate layer 123. Preferably, the intermediate layer 123 should have a thickness substantially ranging from 0.01 mm to 0.05 mm. As a result, when the first lens 121 and/or the second lens 122 is shattered under an external force, the adhesive intermediate layer 123 can adhere the resulting fragments to prevent injury to the user.

The intermediate layer 123 of this invention is made of a transparent material capable of bonding the first lens 121 with the second lens 122 effectively, for example, a liquid adhesive capable of being cured by age hardening, heating or ultraviolet light curing. The intermediate layer 123 may be made of a material selected from the following group: silicon adhesive, polyvinyl acetate adhesive, acrylic adhesive, AB adhesive, cyanoacrylate adhesive, ultraviolet curable adhesive, or a combination thereof.

The first lens 121 and the second lens 122 are respectively made of materials selected from the group consisting of: glass, polycarbonate (PC), polystyrene (PS), polymethylmethacrylate (PMMA), cellulose acetate (CA), cellulose propionate (CP), Acrylonitrile Butadiene Styrene (ABS), Acrylic or the combination thereof. Because the glass has better anti-scratch ability and higher transparency, both the first lens 121 (external lens) and the second lens 122 (internal lens) are made of glass in the first embodiment of this invention. Because the polycarbonate (PC) has better resistance to impact and the glass has higher anti-scratch ability, the first lens 121 (external lens) is made of glass to have the diving mask with better anti-scratch ability while the second lens 122 (internal lens) is made of polycarbonate to have the diving mask with higher impact resistant in the second embodiment of this invention.

Furthermore, the lenses made of cellulose acetate or cellulose propionate has better anti-fog ability than that made of glass, among which the lens made of cellulose acetate has best anti-fog ability. Therefore, in the third embodiment of this invention, the first lens 121 (external lens) is made of glass to have the diving mask with better anti-scratch ability and higher transparency, and the second lens 122 (internal lens) is made of cellulose acetate or cellulose propionate to have the diving mask with better anti-fog ability. Preferably, the second lens 122 is made of cellulose acetate because cellulose acetate has better anti-fog ability than cellulose propionate.

FIG. 3 illustrates the fourth embodiment in accordance with this invention. To further prevent the scattering of fragments, the lens assembly 12 of this invention may further comprise an adhesive film 124, made of a polymer material, to wrap the first lens 121 and the second lens 122 through injection molding, coating or other processes, so that the first lens 121 and/or the second lens 122 is still wrapped by the adhesive film 124 even if they are shattered due to an external impacting force.

In summary, as compared to the safety lens of the prior art, the intermediate layer 123 adopted in this invention is of an adhesive nature, so that the first lens 121 and the second lens 122 may still be adhered thereon without scattering even when they get shattered. Therefore, the lens assembly 12 of this invention can prevent injury to users when the lens assembly shatters during use. Furthermore, the second lens 122 which is made of cellulose acetate has an excellent anti-fog ability and therefore can overcome the inconvenience caused by the fog on the conventional lens.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A lens assembly of a diving mask comprising:
a first lens;
a second lens; and
an intermediate layer, disposed between the first lens and the second lens, in which the intermediate layer is made of a liquid adhesive which is capable of being cured and bonded with the first lens and the second lens.

2. The lens assembly as claimed in claim 1, wherein the liquid adhesive is cured by age hardening, heating or ultraviolet light curing.

3. The lens assembly as claimed in claim 1, wherein the intermediate layer is transparent.

4. The lens assembly as claimed in claim 3, wherein the liquid adhesive is selected from the group of: silicon adhesive, polyvinyl acetate adhesive, acrylic adhesive, AB adhesive, cyanoacrylate adhesive, ultraviolet curable adhesive and the combination thereof.

5. The lens assembly as claimed in claim 4, wherein the intermediate layer has a thickness substantially ranging from 0.01 to 0.05 millimeters.

6. The lens assembly as claimed in claim 1, wherein the first lens and the second lens are respectively made of materials selected from the group consisting of: glass, polycarbonate (PC), polystyrene (PS), polymethylmethacrylate (PMMA), cellulose acetate (CA), cellulose propionate (CP), Acrylonitrile Butadiene Styrene (ABS), Acrylic and the combination thereof.

7. The lens assembly as claimed in claim 6, wherein the first lens is made of glass and the second lens is made of polycarbonate.

8. The lens assembly as claimed in claim 6, wherein the first lens and the second lens are both made of glass.

9. The lens assembly as claimed in claim 6, wherein the first lens is made of glass and the second lens is made of cellulose acetate or cellulose propionate.

10. The lens assembly as claimed in claim 1, further comprising an adhesive film wrapping the first lens and the second lens.
